SEQUENCE LISTING (14 sequences)

SEQ ID NO: 1 - DNA, 4229 bp, Rattus sp.
tcgagtcaactgctcccagatagatccaagacatgagactgtcagcaagaattatttggcttatattatggactgtttgtgtagcagaagattgtaaaggtcctcctccaagagaaaattcagaaattctctcaggttcgtggtctgaacaactatattcagaaggcactcaggcaacctacaaatgccgccctggataccgaacacttggtactattgtaaaagtatgcaagaatggagaatgggtaccttctaacccatcaaggatatgtcggaaaaggccatgtgggcatcccggagacacaccctttgggtcctttaggctggcagttggatctgaatttgaatttggtgcaaaggttgtttatacatgtgatgaagggtaccaactattaggtgaaattgattaccgtgaatgtgatgcagatgggtggaccaatgatattccaatatgtgaagttgtgaagtgcttgccagtgacagaactggagaatggaagaattgtgagtggtgcagccgaaccagaccaggaatattattttggacaggtggtacgctttgaatgcaactccggcttcaagattgaaggacagaaagaaatgcactgctcataaaatggcctctggagcaatgaaaagccacagtgtgtggaaatttcttgcctgccaccacgagttgaaaatggagatggtatatatctgaaaccagtttacaaggagaatgaaagattccaatataaatgtaagcaaggttttgtgtacaaagaaagaggggatgctgtctgcacgggttctggatggaatcctcagccttcctgtgaagaaatgacatgtttgactccatatattccaaatggtatctacacacctcacaggattaaacacagaattgatgatgaaatcagatatgaatgtaaaaatggcttctatcctgcaacccgatcacctgtttcaaagtgtacaattactggctggatccctgctccaagatgtagcttgaaaccttgtgattttccacaattcaaacatggacgtctgtattatgaagaaagccggagaccctacttcccagtacctataggaaaggagtacagctataactgtgacaacgggtttacaacgccttcacagtcatactgggactaccttcgttgcacagtaaatgggtgggagcctgaagttccatgcctcaggcaatgtattttccattatgtggaatatggagaatcttcatactggcaaagaagatatatagagggtcagtctgcaaaagtccagtgtcacagtggctatagtcttccaaatggtcaagatacatattattgtacagagaatggctggtcccctcctcccaaatgcgtccgtatcaagacttgttcagtatcagatatagaaattgaaaatgggtttttttctgaatctgattatacatatgctctaaatagaaaaacacggtatagatgtaaacagggatatgtaacaaataccggagaaatatcaggaataattacttgtcttcaagatggatggtcacctcgaccctcatgcattaagtcttgtgatatgcctgtatttgagaattctatgactaagaataataacacatggtttaaactcaatgacaaattagactatgaatgtcacattggatatgaaaatgaatataaacataccaaaggctctataacatgtacttatgatggatggtctagtacaccctcctgttatgaaagagaatgcagcattcccctgttacaccaagacttagttgtttttcccagagaagtaaaatacaaagttggagattcgttgagtttctcttgccgttcaggacacagagttggagcagatttagtgcaatgctaccactttggatggtcccctaatttcccaacgtgtgaaggccaagtaaaatcatgtgaccaacctcttgaaatcccgaatggggaaataaagggaacaaaaaaagttgaatacagccatggtgacgtggtggaatatgattgcaaacctagatttctactgaagggacccaataaaatccagtgtgttgacgggaagtggacaaggttgccgatatgcgttgagtatgagagaacatgtggagaccttcctgaacttgagcatggctctgtcaagttatctgtccctccctaccatcatggagattcagtggagttcacttgtacagaaaccttcacaatgattggacatgcagtagttttctgcattagtggaaggtggaccgagcttcctcaatgtgttgcaacagatcaactggagaagtgtaaagccccgaagtcaactggcatagatgcaattcatccaaataagaatgaatttaatcataactttagtgtgagttacagatgtagacaaaagcaggagtatgaacattcaatctgcatcaatggaagatgggatcctgaaccaaactgtacaagcaaaagattctgccctcctcccccgcagattccaaatgcccaagtgattgaaaccaccgtgaaatacttggatggagaaaaagtatctgttctttgccaagatggttacctaactcagggcccagaagaaatggtgtgtaaacatggaaggtggcagtcgttaccacgctgcacggaaaaaattccatgttcccagccccctaaaattgaacatggatctattaagtcgcccaggtcctcagaagagaggagagatttaattgagtccagcagttatgaacacggaactacattcagctattgctgtagagatggattcaagatatctgaagaaaatagggtaacctgcaacatgggaaaatggagctctctgcctcgttgtgttggaataccttgtggacccccaccttcaattcctcttggtattgtttctcatgaactagaaagttaccaatatggagaggaggttacatacaattgttctgaaggctttggaattgatggaccagcatttattaaatgtgtaggaggacagtggtctgaacctcccaaatgcataaaaactgattgtgacaacttgcccacatttgaaattgccaaaccgacagaaaagaaaaaaaaatcatacaggtcaggagaacaagtgacattcagatgtccacctccgtatcgaatggatggctctgacattgtcacatgtgttaatacgaagtggattggacagccggtatgcaaagataattcctgtgtgaatccaccacatgtgccaaatgctactatactaacaaggcacaagactaaatatccatctggtgacaaagtacgttatgactgtaataaaccttttgaattatttggggaagtggaagtgatgtgccaaaacgggatttggacagaaccaccgaaatgcaaagattcaacagggaaatgtgggcctcctccacctattgacaatggagacatcacctccttgtcattaccagtatatgcaccattatcatcagttgaatatcaatgccagaactattatctacttaagggaaataagatagtaacatgtagaaatggaaagtggtctcagccaccaacctgcttacatgcatgtgtgataccagaagatattatggaaaaacataatatagttctcagatggagggaaaatgcaaagatttattcccaatcaggggagaatattgaattcatgtgtaaacctggatatagaaaattcagaggatcacctccgtttcgtacaaagtgcattgagggtcacatcaattatcccacttgtgtataaaatcgctatacaattattagtaaaccttatggatgagaaatgcacatgtatattactaatacagtttgaatttacatttaaatattgtttagctcatttcctctaataagtatataaactttttttatatggtggttaatcagtaactttacagactgttgccacaaagcaagaacattacattcaaaactcctaatccaaatatgatatgtccaaggacaaactatgtctaagcaagaaaataaatgttagttcttcaatgtctgtttttattcaggacctttcagattttcttggataccttttgttaggttctgattcacagtgagtggaagacacactgactctgacttcaaattagtattacttgcaatacattaacaaccaaactatcataatatcacaaatgtatacagctaattactgtgtcctacctttgtatcaataaagaaatctaagaaagttcttgcttaaaaaaaaaaaaaaaaaaaa

SEQ ID NO: 2 - DNA, 866 bp, Rattus sp.
tcgagtcaactgctcccagatagatccaagacatgagactgtcagcaagaattatttggcttatattatggactgtttgtgtagcagaagattgtaaaggtcctcctccaagagaaaattcagaaattctctcaggttcgtggtctgaacaactatattcagaaggcactcaggcaacctacaaatgccgccctggataccgaacacttggtactattgtaaaagtatgcaagaatggagaatgggtaccttctaacccatcaaggatatgtcggaaaaggccatgtgggcatcccggagacacaccctttgggtcctttaggctggcagttggatctgaatttgaatttggtgcaaaggttgtttatacatgtgatgaagggtaccaactattaggtgaaattgattaccgtgaatgtgatgcagatgggtggaccaatgatattccaatatgtgaagttgtgaagtgcttgccagtgacagaactggagaatggaagaattgtgagtggtgcagccgaaccagaccaggaatattattttggacaggtggtacgctttgaatgcaactccggcttcaagattgaaggacagaaagaaatgcactgctcataaaatggcctctggagcaatgaaaagccacagtgtgtggaaatttcttgcctgccaccacgagttgaaaatggagatggatatagaaaattcagaggatcacctccgtttcgtacaaagtgcattgagggtcacatcaattatcccacttgtgtataaaatcgctatacaattattagtaaaccttatggatgacactttgtttagaaatgcacatgtatattactaatacagtttgaatttacatttgaaaaa

SEQ ID NO: 3 - DNA, 2715 bp, Rattus sp.
tcgagtcaactgctcccagatagatccaagacatgagactgtcagcaagaattatttggcttatattatggactgtttgtgtagcagaagattgtaaaggtcctcctccaagagaaaattcagaaattctctcaggttcgtggtctgaacaactatattcagaaggcactcaggcaacctacaaatgccgccctggataccgaacacttggtactattgtaaaagtatgcaagaatggagaatgggtaccttctaacccatcaaggatatgtcggaaaaggccatgtgggcatcccggagacacaccctttgggtcctttaggctggcagttggatctgaatttgaatttggtgcaaaggttgtttatacatgtgatgaagggtaccaactattaggtgaaattgattaccgtgaatgtgatgcagatgggtggaccaatgatattccaatatgtgaagttgtgaagtgcttgccagtgacagaactggagaatggaagaattgtgagtggtgcagccgaaccagaccaggaatattattttggacaggtggtacgctttgaatgcaactccggcttcaagattgaaggacagaaagaaatgcactgctcataaaatggcctctggagcaatgaaaagccacagtgtgtgttgaaaccttgtgattttccacaattcaaacatggacgtctgtattatgaagaaagccggagaccctacttcccagtacctataggaaaggagtacagctataactgtgacaacgggtttacaacgccttcacagtcatactgggactaccttcgttgcacagtaaatgggtgggagcctgaagttccatgcctcaggcaatgtattttccattatgtggaatatggagaatcttcatactggcaaagaagatatatagagggtcagtctgcaaaagtccagtgtcacagtggctatagtcttccaaatggtcaagatacatattattgtacagagaatggctggtcccctcctcccaaatgcgtccgtatcaagacttgttcagtatcagatatagaaattgaaaatgggtttttttctgaatctgattatacatatgctctaaatagaaaaacacggtatagatgtaaacagggatatgtaacaaataccggagaaatatcaggaataattacttgtcttcaagatggatggtcacctcgaccctcatgcattaagtcttgtgatatgcctgtatttgagaattctatgactaagaataataacacatggtttaaactcaatgacaaattagactatgaatgtcacattggatatgaaaatgaatataaacataccaaaggctctataacatgtacttatgatggatggtctagtacaccctcctgttatgaaagagaatgcagcattcccctgttacaccaagacttagttgtttttcccagagaagtaaaatacaaagttggagattcgttgagtttctcttgccgttcaggacacagagttggagcagatttagtgcaatgctaccactttggatggtcccctaatttcccaacgtgtgaaggccaagtaaaatcatgtgaccaacctcttgaaatcccgaatggggaaataaagggaacaaaaaaagttgaatacagccatggtgacgtggtggaatatgattgcaaacctagatttctactgaagggacccaataaaatccagtgtgttgacgggaagtggacaaggttgccgatatgcgttgagtatgagagaacatgtggagaccttcctgaacttgagcatggctctgtcaagttatctgtccctccctaccatcatggagattcagtggagttcacttgtacagaaaccttcacaatgattggacatgcagtagttttctgcattagtggaaggtggaccgagcttcctcaatgtgttgcaacagatcaactggagaagtgtaaagccccgaagtcaactggcatagatgcaattcatccaaataagaatgaatttaatcataactttagtgtgagttacagatgtagacaaaagcaggagtatgaacattcaatctgcatcaatggaagatgggatcctgaaccaaactgtacaagcaaaagattctgccctcctcccccgcagattccaaatgcccaagtgattgaaaccaccgtgaaatacttggatggagaaaaagtatctgttctttgccaagatggttacctaactcagggcccagaagaaatggtgtgtaaacatggaaggtggcagtcgttaccacgctgcacggaaaaaattccatgttcccagccccctaaaattgaacatggatctattaagtcgcccaggtcctcagaagagaggagagatttaattgagtccagcagttatgaacacggaactacattcagctattgctgtagagatggattcaagatatctgaagaaaatagggtaacctgcaacatgggaaaatggagctctctgcctcgttgtgttggaataccttgtggacccccaccttcaattcctcttggtattgtttctcatgaactagaaagttaccaatatggagaggaggttacatacaattgttctgaaggctttggaattgatggaccagcatttattaaatgtgtaggaggacagtggtctgaacctcccaaatgcataaaaactgattgtgacaacttgcccacatttgaaattgccaaaccgacagaaaagaaaa

SEQ ID NO: 4 - DNA, 1532 bp, Rattus sp.
tcgagtcaactgctcccagatagatccaagacatgagactgtcagcaagaattatttggcttatattatggactgtttgtgtagcagaagattgtaaaggtcctcctccaagagaaaattcagaaattctctcaggttcgtggtctgaacaactatattcagaaggcactcaggcaacctacaaatgccgccctggataccgaacacttggtactattgtaaaagtatgcaagaatggagaatgggtaccttctaacccatcaaggatatgtcggaaaaggccatgtgggcatcccggagacacaccctttgggtcctttaggctggcagttggatctgaatttgaatttggtgcaaaggttgtttatacatgtgatgaagggtaccaactattaggtgaaattgattaccgttatcgaatggatggctctgacattgtcacatgtgttaatacgaagtggattggacagccggtatgcaaagataattcctgtgtgaatccaccacatgtgccaaatgctactatactaacaaggcacaagactaaatatccatctggtgacaaagtacgttatgactgtaataaaccttttgaattatttggggaagtggaagtgatgtgccaaaacgggatttggacagaaccaccgaaatgcaaagattcaacagggaaatgtgggcctcctccacctattgacaatggagacatcacctccttgtcattaccagtatatgcaccattatcatcagttgaatatcaatgccagaactattatctacttaagggaaataagatagtaacatgtagaaatggaaagtggtctcagccaccaacctgcttacatgcatgtgtgataccagaagatattatggaaaaacataatatagttctcagatggagggaaaatgcaaagatttattcccaatcaggggagaatattgaattcatgtgtaaacctggatatagaaaattcagaggatcacctccgtttcgtacaaagtgcattgagggtcacatcaattatcccacttgtgtataaaatcgctatacaattattagtaaaccttatggatgagaaatgcacatgtatattactaatacagtttgaatttacatttaaatattgtttagctcatttcctctaataagtatataaactttttttatatggtggttaatcagtaactttacagactgttgccacaaagcaagaacattacattcaaaactcctaatccaaatatgatatgtccaaggacaaactatgtctaagcaagaaaataaatgttagttcttcaatgtctgtttttattcaggacctttcagattttcttggataccttttgttaggttctgattcacagtgagtggaagacacactgactctgacttcaaattagtattacttgcaatacattaacaaccaaactatcataatatcacaaatgtatacagctaattactgtgtcctacctttgtatcaataaagaaatctaagaaagttcttgcttaaaaaaaaaaaaaaaaaaaa

SEQ ID NO: 5 - DNA, 27 bp, Artificial Sequence
  Description of Artificial Sequence Oligonucleotide primer
ttcaagtaacgttagaagcttaagatg

SEQ ID NO: 6 - DNA, 33 bp, Artificial Sequence
  Description of Artificial Sequence Oligonucleotide primer
ggcggccgctcaaatcttctgagatataggaga

SEQ ID NO: 7 - DNA, 33 bp, Artificial Sequence
  Description of Artificial Sequence Oligonucleotide primer
ggcggccgctcatttaatccttaaaggtgagta

SEQ ID NO: 8 - DNA, 33 bp, Artificial Sequence
  Description of Artificial Sequence Oligonucleotide primer
ggcggccgctcatactggaaagtatggtctacg

SEQ ID NO: 9 - protein, 207 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKI

SEQ ID NO: 10 - protein, 265 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIK

SEQ ID NO: 11 - protein, 329 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPV

SEQ ID NO: 12 - DNA, 32 bp, Artificial Sequence
  Description of Artificial Sequence Oligonucleotide primer
cctcctcctggaaatgttagaagcttaagatg

SEQ ID NO: 13 - DNA, 29 bp, Artificial Sequence
  Description of Artificial Sequence Oligonucleotide primer
cctctagattacttgatacggacgcattt

SEQ ID NO: 14 - protein, 428 aa, Rattus sp.
EDCKGPPPRENSEILSGSWSEQLYSEGTQATYKCRPGYRTLGTIVKVCKNGEWVPSNPSRICRKRPCGHPGDTPFGSFRLAVGSEFEFGAKVVYTCDEGYQLLGEIDYRECDADGWTNDIPICEVVKCLPVTELENGRIVSGAAEPDQEYYFGQVVRFECNSGFKIEGQKEMHCSENGLWSNEKPQCVEISCLPPRVENGDGIYLKPVYKENERFQYKCKQGFVYKERGDAVCTGSGWNPQPSCEEMTCLTPYIPNGIYTPHRIKHRIDDEIRYECKNGFYPATRSPVSKCTITGWIPAPRCSLKPCDFPQFKHGRLYYEESRRPYFPVPIGKEYSYYCDNGFTTPSQSYWDYLRCTVNGWEPEVPCLRQCIFHYVEYGESSYWQRRYIEGQSAKVQCHSGYSLPNGQDTYYCTENGWSPPPKCVRIK

(19) United States
(12) Patent Application Publication
(10) Pub. No.: US 2006/0178308 A1
(43) Pub. Date: Aug. 10, 2006

(54) Complement inhibitor

(76) Inventors: Wilhelm Schwaeble, Leicester (GB); Robert Braidwood Sim, Kidlington (GB)

Correspondence Address: WALLENSTEIN WAGNER & ROCKEY, LTD, 311 SOUTH WACKER DRIVE, 53RD FLOOR, CHICAGO, IL, 60606, US

(21) Appl. No.: 11/345,903

(22) Filed: Feb. 2, 2006

Related U.S. Application Data
(63) Continuation of application No. 09/316,163, filed on May 21, 1999, now abandoned.
Application No. 09/316,163 is a continuation of application No. PCT/GB97/03275, filed on Nov. 28, 1997.

(30) Foreign Application Priority Data
Nov. 28, 1996 (GB) 9624731.7

(51) Int. Cl. A61K 38/17; C07K 14/74; C07H 21/04; C12P 21/06; C12N 5/06

(52) U.S. Cl. 514/12; 530/350; 435/69.1; 435/320.1; 435/353; 536/23.5

(57) ABSTRACT

The present invention concerns regulation of complement activation, in particular the fluid phase regulation of complement activation, and provides molecules comprising at least complement control protein modules 1-4 of complement factor H, DNA molecules encoding same, their use in the manufacture of a medicament for inhibiting complement activation and methods of same, together with DNA sequences encoding rat FH 4.3 and 1.0 kb mRNA.

### CROSS-REFERENCE TO RELATED APPLICATIONS

This application is a Continuation of co-pending U.S. application Ser. No. 09/316,163, filed May 21, 1999 (CPA filed Jun. 18, 2001), and upon which a claim of priority is based, which application was a continuation of PCT/GB97/03275, filed Nov. 28, 1997, which claimed the benefit of British Application No. 9624731.7, filed Nov. 28, 1996, which applications are incorporated herein by reference and made a part hereof as indicated below.

### FEDERALLY SPONSORED RESEARCH OR DEVELOPMENT

Not applicable.

### BACKGROUND OF THE INVENTION

The present invention concerns regulation of complement activation, in particular the fluid phase regulation of complement activation.

The complement system (see McAleer, M. A. and Sim, R. B. in Activators and Inhibitors of Complement, Kluwer Academic Publishers, Dordrecht, ed R. B. Sim, 1993, p. 1-15; Reid, K. B. M. and Law, A., 1988, Complement, IRL Press, Oxford) is concerned with host defence against infection—upon activation of the system a catalytic set of reactions and interactions occur resulting in the targeting of the activating cell, organism or particle for destruction. Due to the destructive nature of the system it has the potential to cause severe damage to a host system if incorrectly triggered (Davis, A. E., 1988, Ann. Rev. Immunol., 6: 595-628; Frank, M. M., 1993, In: Complement in Health and Disease, 2nd Edition, Whaley, K. et al. eds., Kluwer Academic Publishers, Dordrecht, p. 229) and if its activity is diminished then it has the potential to leave the host open to attack from infecting pathogens.

This is particularly the case with patients suffering from Factor H (FH) deficiency which leads to an uncontrolled activation of the complement system resulting in a depletion of serum complement. Factor H deficient patients are susceptible to recurrent bacterial infection (particularly meningitis) and may not be able to clear immune complexes efficiently from circulation, resulting in glomerulonephritis.

Factor H is an important complement regulator which controls activation by its virtue to bind to native and complexed C3b and to serve as a cofactor in the Factor I mediated conversion of C3b to haemolytically inactive iC3b (Whaley, K. and Ruddy, S., 1976, J. Exp. Med., 144: 1147). It thereby acts as an antagonist to factor B and holds in check the alternative pathway activation, a positive feedback loop in which C3b complexes with factor B, after which the serine protease factor D activates factor B by proteolysis, to form the alternative pathway C3 convertase, C3bBb. Factor H has a further important regulatory function as it can accelerate the decay of the C3 convertase by displacing Bb from the complex (Whaley, K. and Ruddy, S., 1976, Science, 193: 1011). Absence of factor H results in uncontrolled turnover of the alternative pathway. Because C3b is an integral component of the C5 convertases of both classical and alternative pathways, the binding of factor H to C3b also regulates C5 convertase activity (Whaley, K. and Ruddy, S., 1976, Science, 193: 1011). Thus factor H plays a key role in controlling the alternative pathway C3 convertase activity and also the activities of the C5 convertases of both classical and alternative pathways.

No complement regulatory activity has as yet been ascribed to the recently characterized variant factor H related serum glycoproteins of 39/43 kDa and 24/29 kDa (Timmann, C. et al., 1991, J. Immunol., 146:1265; Estaller, C. et al., 1991, J. Immunol., 146: 3190; Schwaeble, W. et al., 1991, Eur J. Biochem., 198: 399-404; Skerka, C. et al., 1991, J. Biol. Chem., 266: 12015; Zipfel, P. F. and Skerka, C., 1994, Immunology Today, 15: 121). These factor H related mRNAs are exclusively expressed in the liver (Schwaeble, W. et al., 1991, Immunobiol., 182:307) and encoded by at least two different factor H related genes (Estaller, C. et al., 1991, J. Immunol., 146: 3190; Hourcade, D. et al., 1991, Abstr. XIVth Int. Complement Workshop, Complement Inflamm., 8: 163; Zipfel, P. F. and Skerka, C., 1994, Immunology Today, 15: 121).

Factor H comprises a number of independently folded domains (CCP modules or short consensus repeats—SCRs) of approximately 60 amino acid (aa) residues with a framework of highly conserved residues involving 4 cysteine, 1 tryptophane and 2 proline residues. In human serum, two different FH glycoproteins of 155 kDa (FHp155) and of 43 kDa (FHp43) are known (Schwaeble, W. et al., 1987, Eur. J. Immunol., 17: 1485; Ripoche, J. et al., 2988, Biochem. J., 249: 593; Schwaeble, W. et al., 1991, Eur. J. Biochem., 198: 399-404; Estaller, C. et al., Eur. J. Immunol., 21: 799) and both forms express cofactor (i.e. complement regulatory) activity in the FI (Factor I) mediated conversion of C3b to iC3b (Misasi, R. et al., 1989, Eur. J. Immunol., 19: 1765-1768). See also Whaley, K. and Ruddy, S., 1976, J. Exp. Med. 144: 1147-1163; Whaley, K. and Ruddy, S., 1976, Science, 193: 1011-1013.

### SUMMARY OF THE INVENTION

According to the present invention there is provided a molecule comprising at least complement control protein (CCP) modules (Reid, K. B. M. et al., 1986, Immunol. Today, 7: 230-234) 1-4 of complement factor H, or a molecule resulting from partial modification thereof or an allelic mutant thereof.

By “partial modification” and “partially modified” is meant, with reference to amino acid sequences a partially modified form of the molecule which retains substantially the properties of the molecule from which it is derived, although it may of course have additional functionality. Partial modification may, for example, be by way of addition, deletion or substitution of amino acid residues. Substitutions may be conserved substitutions. Hence the partially modified molecules may be homologues of the molecules from which they are derived. They may, for example, have at least 40% homology with the molecules from which they are derived. They may for example have at least 50, 60, 70, 80, 90 or 95% homology with the molecules from which they are derived. Similarly nucleotide sequences encoding the molecules or amino acid sequences may be partially modified to code for any such modifications to an amino acid sequence or molecule. Nucleotide sequences may also of course be modified such that they still code for the same amino acid residues but have a different nucleotide sequence.

The molecule may for example comprise CCP modules 1-4,1-5 or 1-6 of complement factor H, or a molecule resulting from partial modification thereof or an allelic mutant thereof.

The present inventor have found that, surprisingly, truncated recombinant factor H expressed in yeast is approximately 10-100 fold more potent (see FIG. 4) than the serum protein FHp155, and that this potency is to be found particularly in constructs representing CCP modules 1-6, CCP modules 1-5, and CCP modules 1-4. For example (FIG. 4) at a 100 nM concentration a 30-40 fold increase in efficacy is observed. This specific potency in CCP modules (SCRs) 1-4, 1-5 and 1-6 has not previousl been suggested or disclosed.

The complement factor H may be human complement factor H or it may for example be a different animal complement factor H, for example rat complement factor H.

The molecule may comprise FHp43, or a molecule resulting from partial modification thereof or an allelic mutant thereof.

The molecule may be for use in inhibiting complement activation.

Hence a molecule according to the present invention may have increased complement inhibitory activity compared to that of FHp155, i.e. it may have an enhanced efficacy. A molecule according to the present invention comprises at least CCP modules 1-4 of FHp43. It may for example comprise at least CCP modules 1-4, 1-5 or 1-6 of FHp43.

A molecule comprising human factor H CCP modules 1-4, 1-5 or 1-6 may have the sequence of SEQ ID NO: 9, 10 or 11 respectively. A molecule comprising rat factor H and having CCP modules 1-7 may have the sequence of SEQ ID NO: 14.

The present inventors have found that the C-terminal 180 amino acids of FHp43 may be removed without significant loss of the complement inhibitory function of FHp43. Hence molecules according to the present invention may have C-terminal deletions of for example about 180 amino acids, when compared to FHp43.

The regulatory activity of these molecules may be used for example in preventing tissue damage due to myocardial infarction, ischemia (for example limb and gut ischemia), infarction of neural tissue, in treating the adult respiratory distress syndrome, rheumatoid arthritis and thermal injuries. The molecules may be used as a fluid phase regulator of complement activity. They may for example be used to improve the biocompatability of artificial membranes by e.g. coating haemofiltration membranes with immobilised FH polypeptides in order to reduce complement activation or by encapsulating xenografts in artificial membranes coated with FH polypeptides. Fusion proteins may be made comprising a FH protein according to the present invention fused to a membrane anchor in order to act as a potent complement regulator on the surface of transfected (or transformed) cells and transgenic animals. Such membrane anchored molecules may be used to reduce xenograft rejection using xenotransplant organs. Spacer residues may be added between the membrane anchor and the FH protein in order to increase or optimise the efficacy of the FH protein (Adams, E. M. et al., 1991, J. Immunol., 147: 3005). Methods of transformation and transfection of cells are well known in the art and where reference is made to transfection, reference is also to transformation and vice versa.

Molecules according to the present invention may be modified such that they have an increased half-life in order that they may have a prolonged protective effect upon a patient. Particular molecules may for example comprise dimeric or trimeric forms of molecules according to the present invention. For example a molecule may comprise a trimer of CCP modules 1-4 or a trimer of FHp43.

Also provided according to the present invention is the use of a molecule according to the present invention in the manufacture of a medicament for inhibiting complement activation. Also provided according to the present invention is a method of manufacture of a medicament for inhibiting complement activation, comprising the use of a molecule according to the present invention.

Also provided according to the present invention is a method of inhibiting complement activation comprising the use of a molecule according to the present invention.

Although human Factor H has previously been clones, researchers have so far failed to clone rat Factor H. The present inventors have now succeeded in isolating and sequencing rat FH 4.3 and FH1.0 mRNA and so according to the present invention there is also provided a nucleotide sequence having the sequence of SEQ ID NO: 1 (FIG. 1—FH4.3) encoding rat FH 4.3 kb mRNA, together with a nucleotide sequence having the sequence of SEQ ID NO: 2 (FIG. 1—FH1.0) encoding rat FH 1.0 kb mRNA. The present invention also extends to partially modified forms of the nucleotide sequences and to polypeptides derived from them and partially modified forms thereof.

FHp155 and FHp43 may be readily isolated and purified (Misasi, R. et al., Eur. J. Immunol., 1989, 19: 1765-1768; Sim, R. B. et al., 1993, Int. Rev. Immunol., 10: 65; Sim, R. B. et al., 1993, Meth. Enzymol., 223: 13 and references therein) and the genes encoding the proteins may be isolated using standard techniques. Standard expression systems, for example MaxBac (Invitrogen) may be used to synthesise the isolated protein (see Sharma, A. K. and Pangbum, M. K., 1994, Gene, 143: 301).

The ability of the molecules of the present invention to inhibit complement activation may be readily shown by activating complement with antigen-antibody complexes (classical pathway) or zymosan (alternative pathway) in the presence of the molecules of the present invention and assaying levels of C3a, C5a and C5b-9 complement components using commercially available reagents (Amersham) and ELISA (enzyme linked immunosorbent assay).

The alternative pathway C3 and C5 convertases ((C3b)ₙBbP) and classical pathway C5 convertase (C4b2a3b) may be readily prepared from for example rat or human components and the activity of the factor H molecules of the present invention on the formation and stability of each convertase and on C5 activation may be assayed using haemolytic assay systems (Sim et al., 1993, supra).

The ability of the molecules of the present invention to inhibit complement activation and limit tissue injury in vivo may be determined using for example a model of perfusion injury of ischaemic myocardium (Weisman, H. F et al., 1990, Science, 249: 146) and a model of antibody-dependent experimental allergic encephalomyelitis (Piddlesden, S. et al., 1990, Clin. Exp. Immunol., 83: 245).

The molecules of the present invention may be readily coupled to artificial membranes, for example dialysis membranes, as follows. Using cuprophan-cellulose membranes (Enka-Azko, Wuppertal, Germany), the following steps may be performed:

i) Activation of the membrane:

1,1′-Carbodiimidazole (Kennedy, J. F. and Paterson, M., 1993, Polymer. Intem., 32: 71;

Chlorformic acid-p-nitrophenylester (Vandorne, F. et al., 1991, Makromol. Chem., 192: 773);

Cyanogen bromide (Kennedy, J. F. and Patterson, M., 1993, supra)

ii) Coupling of spacers:

Use of aliphatic diamines (e.g. 1,12 Diaminododecane, Kery et al., 1991, Carbohydr. Res., 209: 83);

Use of 6-aminocaproicacid (Burton, S. C., 1991, J. Chromatogr., 587: 271);

Use of aminosubstituted aliphatic thiols (Kery et al., 1991, supra)

iii) Coupling of the peptide:

Activation of the N-terminal spacer by thiophosgen;

Activation of a carboxyterminal spacer using alternatively the acid method or the addition of coupling reagents (e.g. DCC or EDC, Royer, G. P. and Anantharmaiah, G. M., 1979, J. Am. Chem. Soc., 101: 3395; Bodanszky, M. and Bodanszky, A., 1984, K. Hafner et al., Hrsg, Bd. 21, Springer-Verlach, Berlin);

Activation of S-terminal spacer by 2,2′-Dithiodipyridine and coupling via cysteine residues.

The effect of uncoated and coated membranes (above) upon complement activation may be readily quantified using C3a, C5a and C5b-9 assays (Chenoweth, D. E., 1987, Contr. Nephrol., 59: 51 and as described above).

According to a further aspect of the invention, there is provided a DNA molecule, which may be in recombinant or isolated form, comprising a sequence encoding a molecule according to the present invention.

The coding sequence may be operatively linked to an expression control sequence sufficient to drive expression. Recombinant DNA in accordance with the invention may be in the form of a vector. The vector may for example be a plasmid, cosmid or phage. A vector may include at least one selectable marker to enable selection of cells transfected (or transformed) with the vector. Such a marker or markers may enable selection of cells harbouring vectors incorporating heterologous DNA. The vector may contain appropriate start and stop signals. The vector may be an expression vector having regulatory sequences to drive expression. Vectors not having regulatory sequences may be used as cloning vectors (as may expression vectors).

Cloning vectors can be introduced into suitable hosts (for example E. coli) which facilitate their manipulation.

According to another aspect of the invention, there is therefore provided a host cell transfected or transformed with DNA according to the present invention. Such host cells may be prokaryotic or eukaryotic. Eukaryotic hosts may include yeasts, insect and mammalian cell lines. Expression hosts may be stably transformed. Unstable and cell-free expression systems may of course also be used.

DNA of the invention may also be in the form of a transgene construct designed for expression in a transgenic plant or animal. In principle, the invention is applicable to all animals, including birds such as placental mammals, (for example cattle, sheep, goats, water buffalo, camels and pigs), domestic fowl, amphibian species and fish species. The protein may be harvested from body fluids or other body products (such as eggs or milk, where appropriate). Such mammalian transgenic mammary expression systems are well known—see for example WO 88/00239, WO 90/05188 and WO 94/16570. The β-lactoglobulin promoter may be used in transgenic mammary expression systems.

Expression hosts, particularly transgenic animals, may contain other exogenous DNA to facilitate the expression, assembly, secretion and other aspects of the biosynthesis of molecules of the invention.

The invention is in principle capable of accommodating the use of synthetic DNA sequences, cDNAs, full genomic sequences and “minigenes”, i.e. partial genomic sequences containing some, but not all, of the introns present in the full length gene.

DNA in accordance with the invention can in principle be prepared by any convenient method involving coupling together successive nucleotides, and/or ligating oligo-and/or poly-nucleotides, including in vitro processes, as well as by the more usual recombinant DNA technology.

### BRIEF DESCRIPTION OF THE DRAWINGS

The invention will be further apparent from the following description, with reference to the several figures of the accompanying drawings, which show, by way of example only, forms of complement inhibition. Of the figures:

FIG. 1 shows sequence alignments of the nucleotide sequences of four different types of rat factor H mRNA transcripts (rFH4.3, rFH2.7, rFH1.8 and rFH1.0; SEQ ID NOs: 1, 3, 4 and 2 respectively). Start and stop-codons are underlined, the polyadenylation initiation signal is written in italics;

FIG. 2 shows a cofactor assay showing the functional activity of recombinant human FHp43. Lanes are as follows: Lane 1-C3b with human Factor I (FI); lane 2-C3b with rat FI; lane 3-C3b with human FI and recombinant rat FHSCR1-7; lane 4-C3b with human FI and recombinant human FHp43 (10 mM); and lane 5-C3b with rat FI and purified human factor H;

FIG. 3 shows a cofactor assay showing the functional activity of recombinant rat FHSCR1-7. Lanes are as follows: Lane 1-C3b with human FI; lane 2-C3b with rat FI; lane 3-C3b with human FI and recombinant human factor H; lane 4-C3b with human FI and recombinant rat factor H; lane 5-C3b with rat FI and recombinant rat FHSCR1-7; lane 6-C3b with rat factor 1 and 10 mM recombinant rat FHSCR1-7; and lane 7-C3b with human factor 1 and 10 mM recombinant FHp43; and,

FIG. 4 shows the results of a cofactor assay performed to compare the functional activity of truncated recombinant human factor H SCR1-4, SCR1-5 and SCR1-6 with that of purified serum FHp155. The values given are arbitrary values representing the relative abundance of the 43 kDa C3b cleavage product obtained by the factor I-mediated cleavage of ¹²⁵I-labelled C3b using densitometry. COncentration of purified recombinant and native factor H proteins added to the assay are given in the left column.

### DETAILED DESCRIPTION

### EXPERIMENTAL

With the following experiments, a truncated recombinant human and rat factor H are expressed in a high efficiency yeast expression system. The yield of expression is estimated to be in a range of up to 5 mg of recombinant protein per litre of yeast culture.

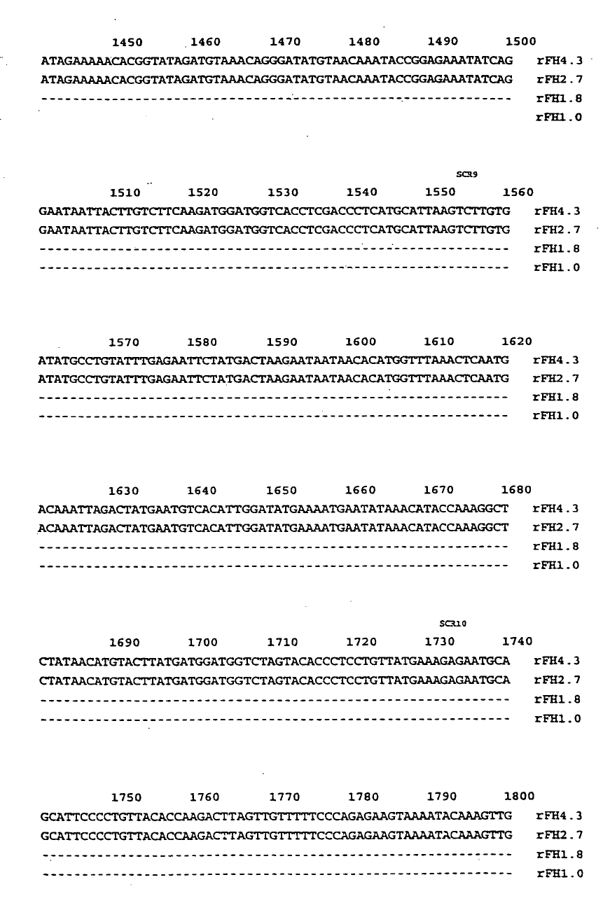

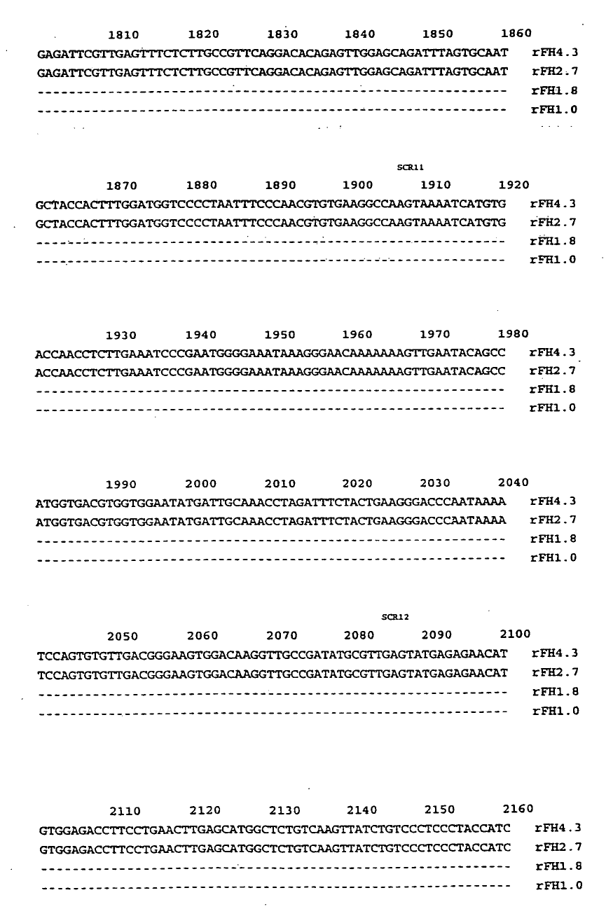

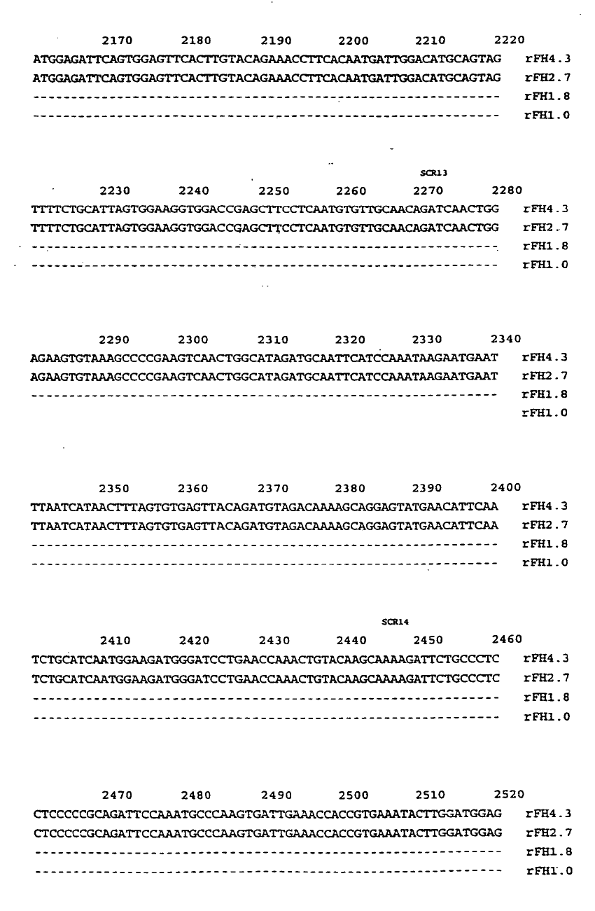

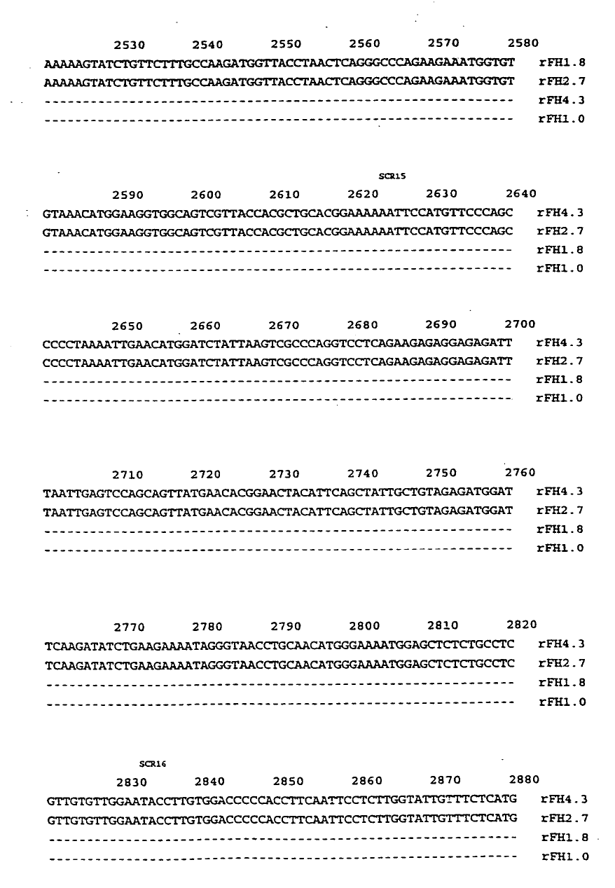

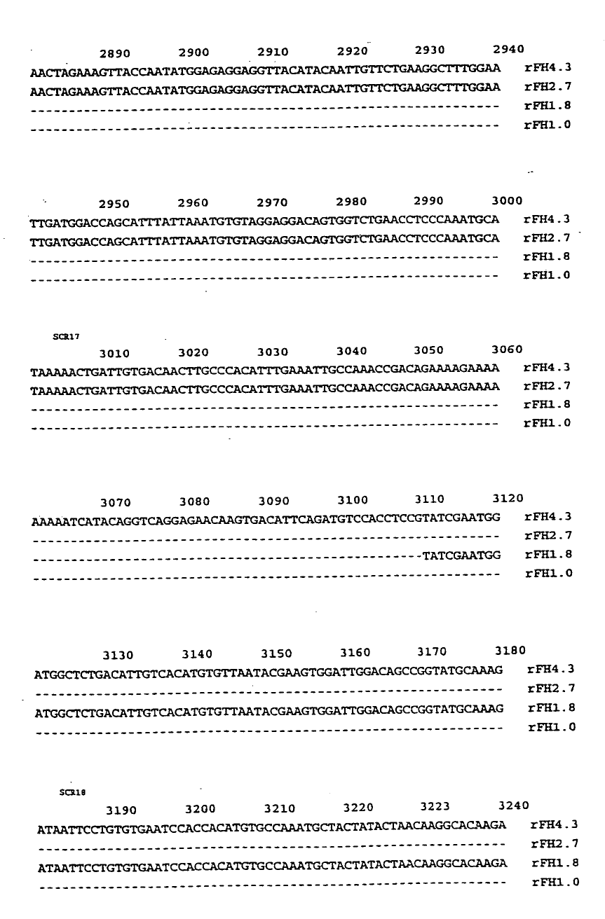

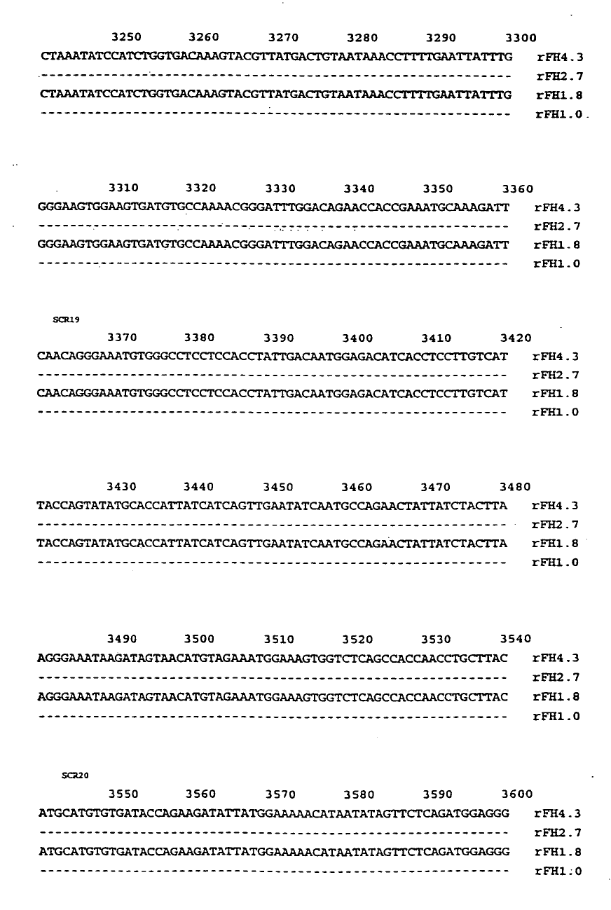

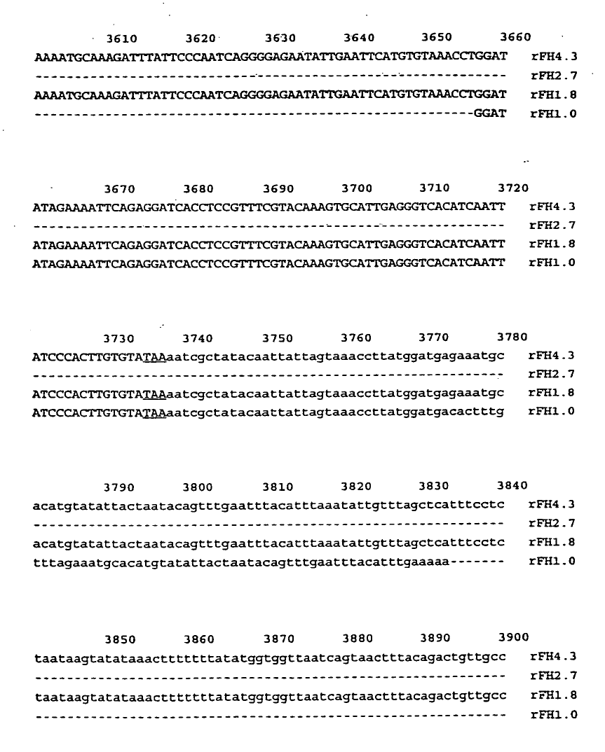

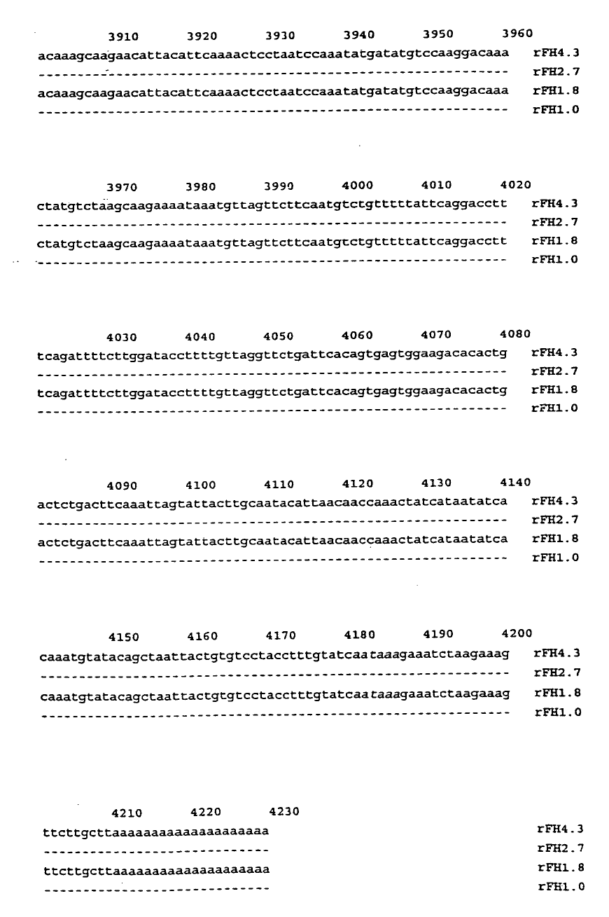

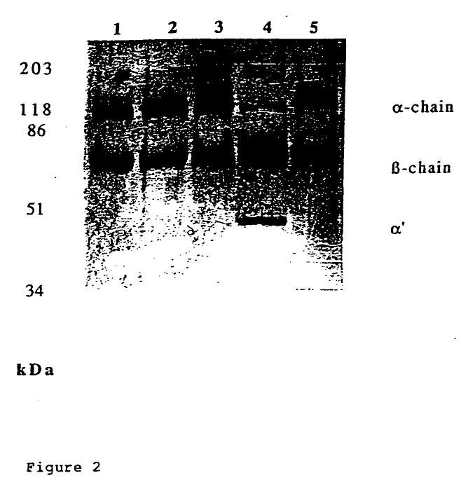

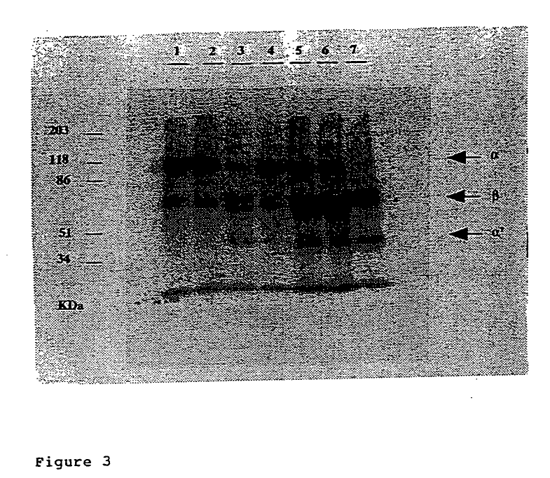

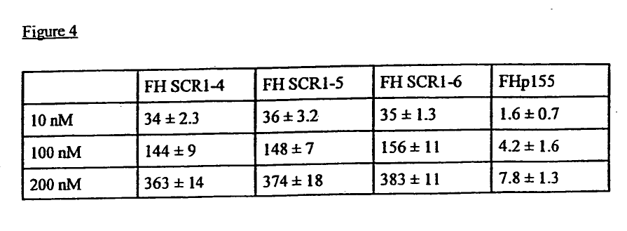

FIGS. 2 and 3 show the results of the cofactor assays described below. The presence of an a′ band at 43 kDa (a cleavage product of the α-chain of C3b) indicates cofactor activity (FIG. 2, lane 4; FIG. 3, lanes 3, 5, 6 and 7). Hence both the recombinant human FHp43 and rat FHSCR1-7 peptides cooperate with factor I in a species specific manner and, surprisingly, exhibit cofactor activity even at low concentrations (10 mM) when incubated with C3b and factor I of the corresponding species.

Materials and Methods

Isolation and Characterization of 4 Different Factor H or Factor H Related Gene Products of the Rat

Using a rat liver cDNA library in λ-ZAP II (#937506 STATAGENE, La Jolla, Calif.), cDNA clones rFH4.3, rFH1.8, rFH2.7 and rFH1.0 were isolated as follows. Approximately 300,000 colonies were screened with a 5′ specific PstI/XhoI cDNA subfragment of the mouse factor H cDNA clone MH8 (Kirstensen, T. et al., 1986, J. Immunol., 136: 3407). From eighteen hybridizing plaques obtained in the rescreen procedure, the four clones listed above were analysed further. The pBluescript SK− plasmid containing the cDNA insertions of interest were rescued from the λ-ZAP II phagemid by in vivo excision. The cDNA sequences of the 4 different types of clones was determined by sequencing both strands using the Sanger dideoxy chain termination method with Sequenase II (RTM) and the reagent kit (USB, Cleveland, USA).

RNA Extraction and Northern Blot Analysis

Total RNA was isolated according to standard methods (Chirgwin, J. W. et al., 1979, Biochemistry, 18: 5294), quantified by measuring the absorbence at 260 nm, separated on a formaldehyde-containing 1.2% agarose gel and blotted to Hybond N filters. Agarose gel electrophoresis, RNA transfer and hybridization of blots were performed by standard techniques (Sambrook, J., Frisch, E. F., and Maniatis, T.: Molecular cloning. A laboratory manual, Cold Spring Harbor Laboratory, Cold Spring Harbor Press, New York, 1989). Northern blot filters were probed with a 5′-specific 553 bp long PstI/XhoI restriction subfragment of the murine factor H clone MH8 encoding SCR 1-2 of mouse factor H, and the 867 bp long cDNA insert of the rat specific factor H clone rFH1.0. The probes were used at a concentration of 5×10⁶ cpm of ³²p labelled cDNA/ml hybridization solution. Hybridization was performed at 65° C. in the absence of formamide. The washing of the Northern blots was carried out according to standard procedures (Sambrook et al., 1989, supra). The last washing step was performed in 0.3×SSC for 1 hour at 65° C.

In order to obtain recombinant proteins of lower molecular weight than the naturally occuring factor H serum proteins and in order to opimise complement regulatory activity, we have modified the coding sequence of the human 1.8 kb factor H mRNA sequence. The modifications include a linker sequence insertion to enable an in frame cloning of the first codon for position 1 of the mature factor H protein with the coding sequence of the α-secretory factor contained in the yeast expression vector as well as the insertion of translation termination codons in order to obtain truncated forms of recombinant human factor H. Three constructs of different length were produced, encoding the SCR-motifs 1-4, SCR1-5, and SCR1-6. The pairs of oligonucleotide primers used to amplify the different stretches of coding sequence for human factor H (Schwaeble, W. et al., 1987, Eur. J. Immunol., 17: 1485; Ripoche, J. et al., 1988 Biochem. J., 249: 593; Schwaeble, W. et al., 1991, Eur. J. Biochem., 198: 399-404; Estaller, C. et al., Eur. J. Immunol., 21: 799) by PCR are listed below.

For the construct encoding SCR 1-4:

Forward primer (sense orientation)

(SEQ ID NO: 5)

3′ gta gaa ttc GAA GAT TGCAAT GAA CTT 5′
Reverse primer (ligates and introduces a stop
codon at the end of the coding sequence for
SCR4, anti-sense orientation)

(SEQ ID NO: 6)

3′ AGA GGA TAT AGA GTC TTC TAA ACT cgc cgg cgg 5′

For the construct encoding SCR 1-5:

Forward primer (sense orientation)

(SEQ ID NO: 5)

3′ gta gaa ttc GAA GAT TGCAAT GAA CTT 5′
Reverse primer (ligates and introduces a stop
codon at the end of the coding sequence for
SCR5, anti-sense orientation);

(SEQ ID NO: 7)

3′ ATG AGT GGA AAT TCC TAA TTT ACT cgc cgg cgg 5′

For the construct encoding SCR 1-6:

Forward primer (sense orientation)

(SEQ ID NO: 5)

3′ gta gaa ttc GAA GAT TGCAAT GAA CTT 5′
Reverse primer (ligates and introduces a stop
codon at the end of the coding sequence for
SCR6, anti-sense orientation)

(SEQ ID NO: 8)

3′ GCA TCT GGT ATG AAA GGT CAT ACT cgc cgg cgg 5′

Each of the three different PCR products was digested with the restriction endonucleases EcoRI and NotI and subcloned in the polylinker region of the EcoRI/NotI digested yeast expression vector pPICZαA (Invitrogen BV, Leek, The Netherlands). Plasmids were grown in the E. coli strain TOP10F and sequenced to confirm the in frame cloning and the absence of cloning artifacts within the coding sequence. These constructs were used to transfect Pichia Pastoris host cells (strain SMD 1168), transformants selected on YPD/Zeocin agar and genomic transmission of the constructs tested by PCR. Expression of the constructs was performed according to the manufacturer's protocol

The three different constructs therefore encode recombinant proteins representing different parts of the N-terminal sequence of human factor H

The protein sequence of the truncated recombinant human factor H protein SCR1-4 (a protein of 207 aa and 23 kDa) is SEQ ID NO: 9.

The protein sequence of the truncated recombinant human factor H protein SCR1-5 (a protein of 265 aa and 30 kDa) is SEQ ID NO: 10.

The protein sequence of the truncated recombinant human factor H protein SCR1-6 (a protein of 329 aa and 37 kDa) is SEQ ID NO: 11

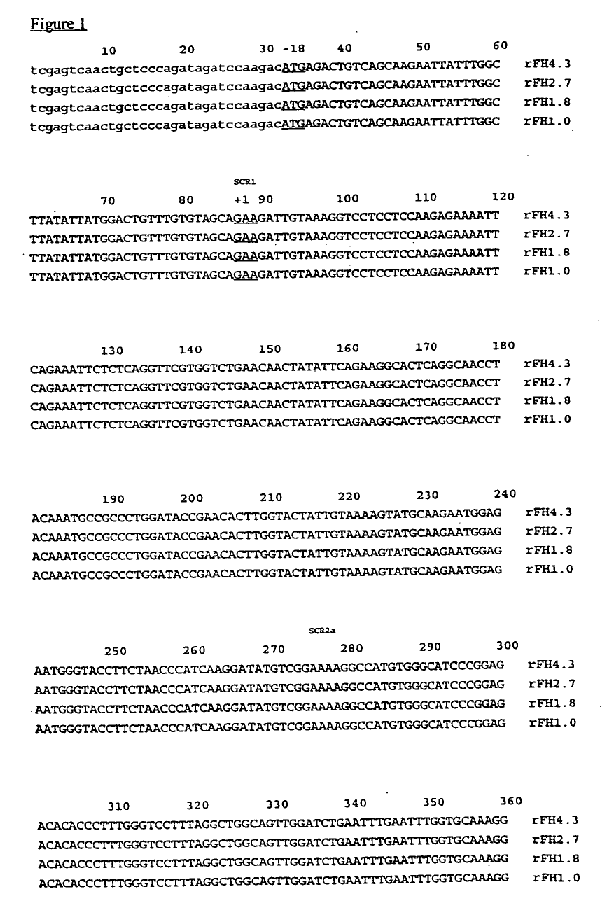

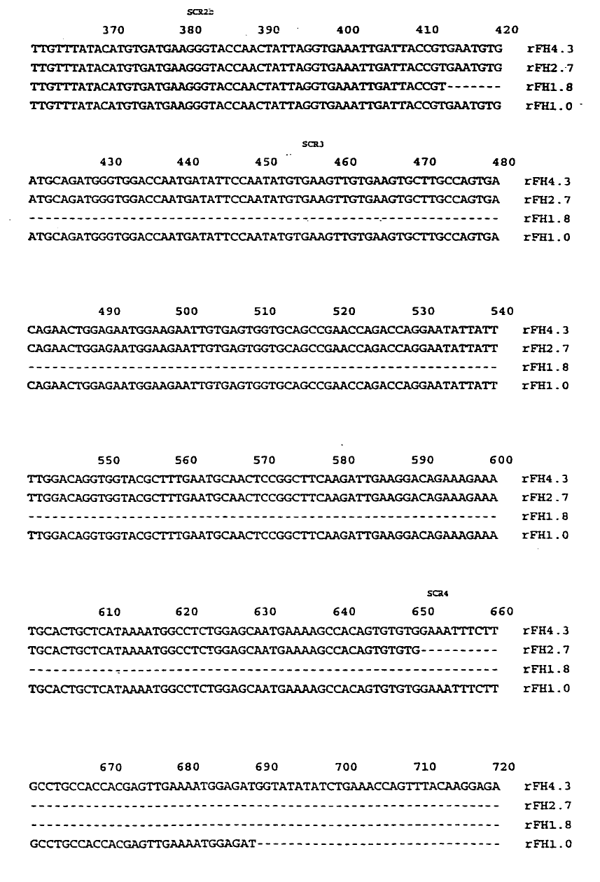

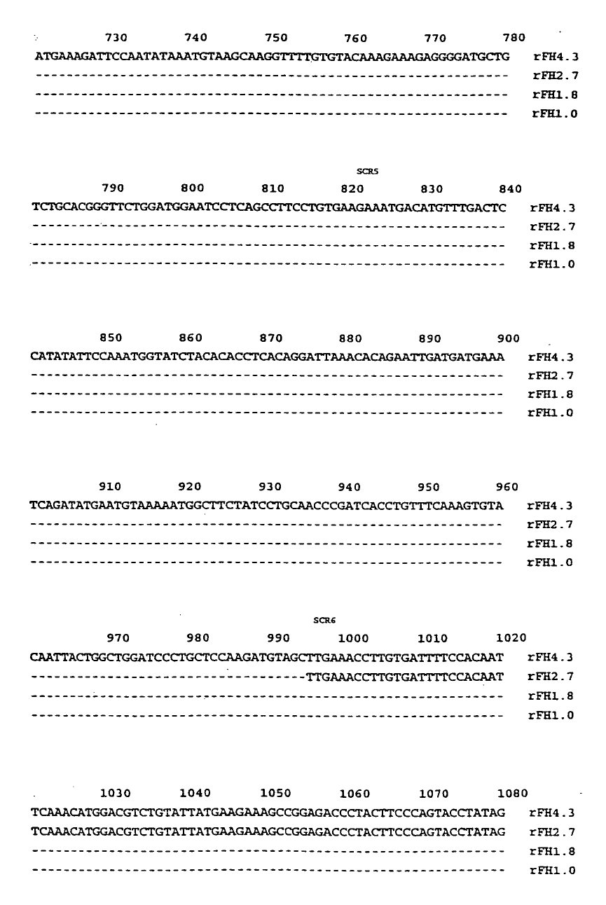

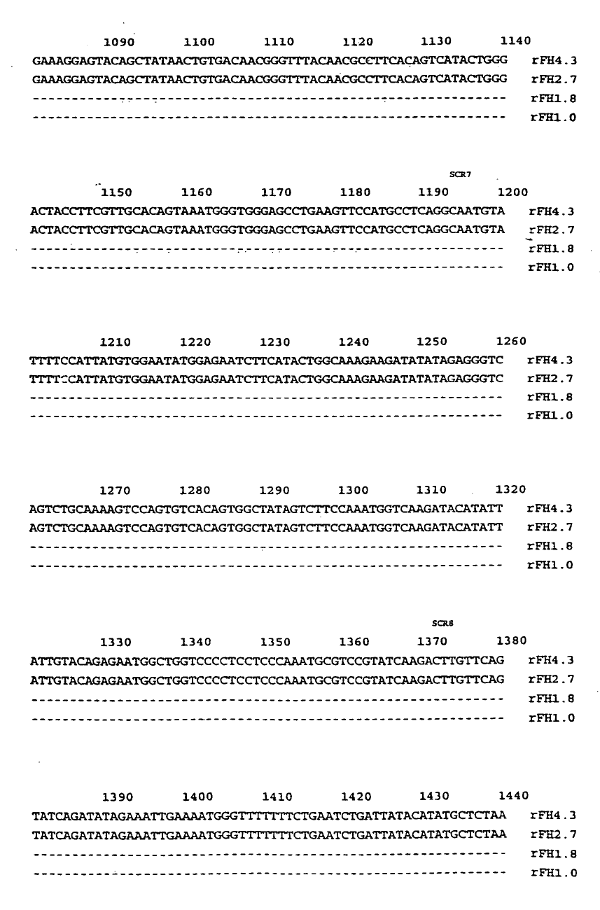

In order to provide reagents that can be used to assess the therapeutic potential of recombinant factor H in rat experimental animal models, a truncated recombinant protein for rat factor H was prepared taking advantage of our rat factor H cDNA for FH4.3 (shown in FIG. 1 below):

As the functionally relevant SCR domains of rat factor H have not yet been mapped precisely, we expressed a slightly larger protein representing the 7 N-terminal SCR domains.

The following oligonucleotides were used to construct the

cDNA Encoding Rat Factor H SCR 1-7:

Forward primer (sense orientation)

(SEQ ID NO: 12)

3′ gta gaa ttc GAA GAT TGT AAA GGT CCT CCT CC 5′
Reverse primer (ligates and introduces a stop
codon at the end of the coding sequence for
SCR7, anti-sense orientation)

(SEQ ID NO: 13)

3′ TTT ACG CAG GCA TAG TTC ATT aga tct cc 5′

The PCR product was digested with the restriction endonucleases EcoRI and XbaI and subcloned in the polylinker region of the EcoRI/XbaI digested yeast expression vector pGAPZαA (Invitrogen BV, Leek, The Netherlands). Plasmids were grown in the E. coli strain TOP10F and sequenced to confirm the in frame cloning and the absence of cloning artifacts within the coding sequence. These constructs were used to transfect Pichia Pastoris host cells (strain SMD 1168), transformants selected on YPD/Zeocin agar and genomic transmission of the constructs tested by PCR. Expression of the constructs was performed according to the manufacturer's protocol. After electroporation, Pichia pastoris cells were plated on MD plates (containing dextrose) and grown at 30% C for 48 hours. Single colonies were picked from these plates and replated on Methanol containing MM plates (without dextrose) to select for AOX1-disrupted transformants which have the cDNA of interest inserted into the polylinker region. Alcohol oxidase genes AOX1 and AOX2 allow the metabolism of methanol, thereby providing a source of carbohydrates. MM plates (without dextrose) provide no other source of carbohydrates and so AOX1-disrupted transformants, which have a reduced ability to metabolise methanol, were recognised by their slower growth on dextrosol-free MM plates. The insertion of the cDNA construct of interest was further confirmed by PCR analysis of genomic DNA isolated from poorly growing colonies. In order to select for such colonies that secrete high rates of recombinant factor H, twenty AOX1-disrupted colonies were inoculated each in 10 ml of BMGY medium (Invitrogen) in a 50 ml tube and cultured at 30° C. with vigorous shaking (>200 rpm) for 48 hours to saturation (OD₆₀₀=10.0-20.0). Cells were harvested by centrifugation for 10 minutes at room temperature at 4000 g, supernatant discarded and the pellet resuspended in 2 ml of BMMY (Invitrogen) medium. This time, tubes were only covered with two layers of sterile gauze and again, incubation occurred at 30° C. with vigorous shaking (>200 rpm) for 48 hours. Cells were pelleted as before and supernatants analysed by Western blot analysis.

The protein sequence of the truncated recombinant rat factor H protein SCR1-7 (a protein of 428 aa and 49 kDa) is SEQ ID NO: 14

After induction of expression, supernatants from all of the 4 different constructs were run through an ion exchange column an the recombinant factor H proteins purified of C1-4B sepharose coupled to polyclonal anti human or polyclonal anti-rat antibodies.

The recombinant truncated rat and human factor H proteins were assessed for complement regulatory activity and compared with purified serum factor H using a factor H dependent cofactor assay.

Cofactor Assay

Functional activity of recombinant rat and human factor H was determined in a factor H dependent factor I mediated C3b cleavage assay. Therefore, human C3b and factor I were purified from peripheral blood as previously described (Misasi, R. et al., 1989, Eur. J. Immunol., 19: 1765). In order to establish a species-specific variant of this assay, rat factor I was purified from 2 ml of rat serum by fluid phase liquid chromatography using Pharmacia FPLC apparatus P500 and a Pharmacia Mono S HR 5/5 column eqilibrated with PE buffer at pH 6. Separation of serum proteins occurred by addition of PE-buffer plus 1M NaCl at pH 6 and a flow rate of 1 ml/min. Fractions were depleted of factor H by immune-chromatography using a Sepharose C14b column preabsorbed with the human anti-factor H monoclonal antibody OX23 (Schwaeble, W. et al., 1987, Eur. J. Immunol., 17: 1485). Human C3 and factor I were prepared from human serum as described earlier (Hammer, C. H.; Wirtz, G. H.; Renfer, L.; Gresham, H. D.; and Tack, B. F. J. Biol. Chem. 1981, 256: 3995; Lambris, J. D. Dobson, N. J.; and Ross, G. D. J. Exp. Med. 1980. 152: 1625. C3b was prepared by limited tryptic digestion of C3 (Bokisch V. A.; Müller-Eberhard, H. J.; and Cochrane, C. G. J. Exp. Med. 1969. 129: 1109) and consecutive chromatography on Sephadex G-100 (equilibrated in 10 mM sodium phosphate/150 mM NaCl buffer, pH7.3) This preparation was radiolabelled with ¹²⁵I (1mCi037 MBq of Na ¹²⁵I per 200 μg C3b) by the Iodogen method (Iodobeads purchased from Pierce Chemical Co. Rockford, Ill.) The specific activity was about 10⁶ cpm/μg C3b. In the assay procedure 300 000 cpm of ¹²⁵I-labelled C3b was mixed with increasing concentrations of recombinant human factor H proteins FH1-4, FH 1-5 and FH1-6 and serum factor H and 0.2 μg of purified human factor I in PBS containing 2 mM DFP in a total volume of 100 μl and incubated for 30 minutes at 37° C. Cleavage of C3b was monitored by SDS-PAGE and autoradiography by the generation of the 73 kDa and 43 kDa cleavage products of the α-chain of C3b. Production of the 43 kDa cleavage product was indicative of cofactor activity.

Samples were analysed by SDS-PAGE under reducing conditions on a 9.5% SDS gel. Gels were dried and finally exposed to autoradiography on X-ray films.

1. A molecule comprising at least complement control protein modules 1-4 of rat complement factor H, or a molecule resulting from partial modification thereof, or an allelic mutant thereof.

2. A molecule according to claim 1 comprising complement control protein modules 1-4, 1-5 or 1-6 of complement factor H, or a molecule resulting from partial modification thereof, or an allelic mutant thereof.

3. A molecule according to claim 1, comprising complement control protein modules 1-7 and having the sequence of SEQ. ID NO: 14.

4. A molecule according to claim 1, for use in inhibiting complement activation.

5. A module according to claim 4, having an enhanced efficacy when compared to FHp155.

6. A method of manufacture of a medicament for inhibiting complement activation, comprising the use of a molecule according to claim 1.

7. A method of inhibiting complement activation comprising the use of a molecule according to claim 1.

8. A nucleotide sequence having the formula of SEQ. ID NO: 1 and encoding rat FH 4.3 kb mRNA.

9. A nucleotide sequence having the formula of SEQ ID NO: 2 and encoding rat FH 1.0 mRNA.

10. A DNA molecule comprising a sequence encoding a molecule according to claim 1.
